(19) United States
(12) Patent Application Publication
(10) Pub. No.: US 2005/0079231 A1
(43) Pub. Date: Apr. 14, 2005

(54) Use of ingested sublimed sulfur for the restoration of lactose tolerance in humans with the medical disorder of lactose intolerance

(76) Inventor: Airudin S. Khan, London (CA)

Correspondence Address: Dr. Airudin S. Khan, 825 Talbot Street, London, ON, N6A 2V7, CA

(21) Appl. No.: 10/942,416

(22) Filed: Aug. 24, 2004

Related U.S. Application Data
(60) Provisional application No. 60/517,891, filed on Nov. 7, 2003.

(30) Foreign Application Priority Data
Oct. 3, 2000 (CA) 2,265,921
Apr. 8, 1999 (CA) 60/128358

(51) Int. Cl. A61K 33/04

(52) U.S. Cl. 424/705

(57) ABSTRACT

From May 1998 through April 2002, 41 patients (22 with clinical and 19 with both clinical and laboratory evidence of severe lactose intolerance) were treated with sublimed sulfur (USP) and a restricted lactose diet for a mean of 3.6 (standard deviation [SD] 3.4) weeks, followed by a regular diet and sublimed sulfur for a mean of 5.8 (SD 5.0) weeks. Quantitative comparisons were made between baseline and post-treatment symptoms in all patients and standard lactose tolerance test results in a subset of 19 patients with positive baseline lactose tolerance tests.

International Application Number: 60/128 358 Apr. 08, 1999

Canadian Patent Number: 2,265,921 Oct. 03, 2000

U.S. patent application Ser. No.: 60/517,891 Nov. 07, 2003

From May 1998 through April 2002, 41 patients (22 with clinical and 19 with both clinical and laboratory evidence of severe lactose intolerance) were treated with sublimed sulfur (USP) and a restricted lactose diet for a mean of 3.6 (standard deviation [SD] 3.4) weeks, followed by a regular diet and sublimed sulfur for a mean of 5.8 (SD 5.0) weeks. Quantitative comparisons were made between baseline and post-treatment symptoms in all patients and standard lactose tolerance test results in a subset of 19 patients with positive baseline lactose tolerance tests.

### RESULTS

Of the 41 patients, 39 (95%) became asymptomatic during treatment, (2 patients were lost to follow-up). Of the remaining 39, 20 (51%) remained symptom-free after one course of treatment. Their overall relapse rate was 0.46 (confidence interval 0.32-0.61).

Of the 19 patients whose symptoms redeveloped, 2 more were lost to follow-up; 13 (76% of 17) remained asymptomatic after a second course of treatment. The median symptom-free period after discontinuation of sublimed sulfur was 76 weeks. In the subset of 19 patients, 17 (89%) became clinically asymptomatic after treatment, and their serum glucose levels increased significantly above baseline at 30 (t=2.81;p=0.01) and 60 minutes (t=2.58;p=0.02) of the lactose tolerance test; 9 (47% of 19) converted from intolerant to tolerant test results.

### CONCLUSION

A limited course of treatment with sublimed sulfur caused a significant increase in lactase activity and restored lactose tolerance for prolonged periods in patients with clinical lactose intolerance.

1. A regimen of treatment with Sublimed Sulfur restores lactose tolerance in patients with lactose intolerance

2. The lactose tolerance as defined in claim 1 persists while treatment with Sublimed Sulfur is continued.

3. The lactose tolerance as defined in claim 1 and claim 2 persists after Sublimed Sulfur is discontinued.

4. A regimen of treatment with Sublimed Sulfur, as defined in claim 1, claim 2 and claim 3, restores lactose tolerance again in patients who suffer a relapse and develop lactose intolerance after Sublimed Sulfur is discontinued.
